(19) United States
(12) Patent Application Publication
(10) Pub. No.: US 2017/0209476 A1
(43) Pub. Date: Jul. 27, 2017

(54) Treatment of Glycosylation Deficiency Diseases

(71) Applicant: Wellstat Therapeutics Corporation, Rockville, MD (US)

(72) Inventor: Reid W. VON BORSTEL, Potomac, MD (US)

(73) Assignee: Wellstat Therapeutics Corporation, Rockville, MD (US)

(21) Appl. No.: 15/329,647

(22) PCT Filed: Aug. 19, 2015
(86) PCT No.: PCT/US2015/045896

§ 371 Date: Jan. 27, 2017

Related U.S. Application Data
(60) Provisional application No. 62/039,052, filed on Aug. 19, 2014.

(51) Int. Cl. A61K 31/7072; A61K 45/06

(52) U.S. Cl. CPC A61K 31/7072; A61K 45/06

(57) ABSTRACT

Uridine triacetate or other uridine prodrugs are used to treat genetic glycosylation disorders by administering them in an amount sufficient to raise plasma uridine in the to a level greater than 30 micromolar. They can be administered alone or in combination with a sugar whose transfer is defective in the glycosylation disorder being treated.

### BACKGROUND OF THE INVENTION

Congenital disorders of glycosylation (CDG) encompass a group of genetic diseases caused by defects in enzymes of glycosylation pathways mediating synthesis of oligosaccharides on glycoproteins or glycolipids. Many glycosylation reactions use uridine diphosphate (UDP) as a sugar transfer molecule, with intermediates such as UDP-glucose, UDP-glucosamine, UDP-galactose, UDP galactosamine, UDP-mannose, UDP-mannosamine and other nucleotide sugars. Such glycosylation disorders include but are not limited to those reviewed in Freeze H (2013) JBC 288(210):6936-6945. Additional specific glycosylation disorders are identified frequently, as analytical and diagnostic techniques improve. Glycosylation defects addressable by the methods and compositions of this disclosure can comprise deficits in synthesis of specific sugars or sugar nucleotides, or in deficits in enzymes catalyzing glycosylation reactions. Glycosylation defects can be due to activity-reducing mutations affecting enzyme structure and function or to reduced expression of active enzymes.

### SUMMARY OF THE INVENTION

This invention provides a method of treating a genetic glycosylation disorder in a subject, comprising administering to the subject a uridine prodrug in an amount sufficient to raise plasma uridine in the subject to a level greater than 30 micromolar. This invention provides a uridine prodrug for use in treating a genetic glycosylation disorder in a subject, wherein the uridine prodrug is administered to the subject in an amount sufficient to raise plasma uridine in the subject to a level greater than 30 micromolar. This invention provides the use of a uridine prodrug in the manufacture of a medicament for treating a genetic glycosylation disorder in a subject, wherein the medicament is formulated for administration in an amount sufficient to raise plasma uridine in the subject to a level greater than 30 micromolar. This invention provides a pharmaceutical composition for use in treating a genetic glycosylation disorder in a subject, the composition comprising a uridine prodrug in an amount sufficient to raise plasma uridine in the subject to a level greater than 30 micromolar.

### DETAILED DESCRIPTION OF THE INVENTION

In diseases with a defect in a pathway in which a glycosylation reaction involving a UDP-sugar (or in the case of sialic acid, a cytidine monophosphate (CMP)-sugar) are at fault, production of the deficient nucleotide sugar (pyrimidine-phospho-sugar) via residual, but inadequate, enzyme activity is augmented by precursor loading, i.e. increasing intracellular uridine triphosphate (UTP) and the specific sugar or its precursor, to form increased intracellular concentrations of the UDP-sugar.

Extracellular uridine concentrations sufficient to increase intracellular UTP and UDP sugars are required, and are in the range of >30 micromolar steady-state concentration, advantageously >50 micromolar while at the same time increasing availability of the specific sugar. Sugar availability may be increased by either administration of the specific sugar or a precursor that is efficiently converted to it, or by inhibiting pathways involved in sugar degradation, or both administering a sugar or precursor and blocking degradation pathways.

Uridine is advantageously administered to patients in the form of an orally bioavailable prodrug such as uridine triacetate (2′,3′,5′-tri-O-acetyluridine). An appropriate single dose is in the range of 2 to 10 grams, administered once to four times daily depending on need and response.

The specific sugars, examples of which are glucosamine, N-acetylglucosamine, galactose, N-acetylgalactose, mannose, or N-acetylgalactose, precursors of these or other relevant sugars, or inhibitors of their catabolism, are administered in doses sufficient to raise their intracellular concentrations in tissues affected by the specific glycosylation disorder.

CDG often affect the development and function of the brain, but also frequently affect peripheral organs as well. A wide variety of symptoms can be caused by glycosylation disorders, including developmental delays (both cognitive and physical), seizures, ataxia, hypotonia, seizures, retinal problems, liver abnormalities including fibrosis, impaired hematopoiesis, and structural malformations. Definitive diagnosis generally requires identification of a pathogenic mutation in a glycosylation pathway, which provides the requisite information for determining whether uridine loading is likely to be beneficial.

CDG have classically been divided into two main groups based on diagnosis using transferrin glycosylation patterns. Type I CDG affects the early steps in oligosaccharide synthesis, production of lipid-linked oligosaccharides in the endoplasmic reticulum. Type II glycosylation disorders comprise defects in glycosylation that occur after glycans are added to proteins, and affect subsequent elongation, trimming and processing of the attached glycans. More recently, CDG that do not fit into either of these categories have been identified, affecting non-glycan glycosylation of proteins. See Freeze H (2013) JBC 288(210):6936-6945, and the references cited therein, all of which are incorporated herein by reference. Pyrimidine-phospho-sugar precursors are involved in subsets of both Type I and Type II CDG, and in glycosylation disorders not falling into either of these categories which will be referred to herein as Type III glycosylation disorders. (“Type I” CDG and “Type II” CDG are art-recognized names for these categories of CDG, whereas the name “Type III” CDG is used in this document for convenience.) PGM deficiency (see below and example 3) and GPT deficiency (see example 4) are examples of Type II CDG. GNE myopathy (see below and example 2) is primarily a Type II CDG but also extends to Type III CDG.

The typical dose for uridine prodrugs in general and uridine triacetate in particular is an amount sufficient to achieve steady state average plasma concentration >30 micromolar, but levels as high as about 50 micromolar (e.g., from 45 to 55 micromolar) may be necessary for some difficult patients. Appropriate doses of uridine triacetate to achieve steady state plasma uridine >30 micromolar are 0.5 to 5 grams per square meter of body surface area (BSA), advantageously 1.5 to 3 grams per square meter of BSA. BSA is determined using standard drug dosing tables or formulas, using body weight, height, sex and age as input variables. Doses are administered orally one to four times per day, approximately evenly spaced throughout the 24 hour day.

The distinguishing characteristic of CDG that are beneficially treated with a uridine precursor and sugar or sugar precursor are those in which the biochemical lesion comprises a deficiency in production or availability (including transport deficits reducing pyrimidine nucleotide sugar delivery into the endoplasmic reticulum) of a pyrimidine nucleotide sugar, or those in which enzyme activity using a pyrimidine nucleotide sugar as a substrate are low, and thereby contributing to disease pathogenesis.

Glucosamine (UDP-N-acetyl)-2-epimerase/N-acetylmannosamine kinase (GNE) is an enzyme involved in synthesis of sialic acid, a sugar found in many glycoproteins. GNE myopathy, or hereditary inclusion body myopathy is caused by a defect in production of sialic acid, a precursor for synthesis of sialoglycoconjugates. Exogenous sialic acid (or sialic acid prodrugs or precursors such as mannosamine or N-acetylmannosamine) is being tested as a treatment, bypassing the enzymatic defect. The agents and methods of this disclosure augments the efficacy of exogenous sialic acid by increasing intracellular pyrimidine nucleotides, including cytidine triphosphate (CTP), which is derived from uridine triphosphate (UTP). Increased intracellular CTP improves the efficiency of conversion of exogenous sialic acid to CMP-sialic acid, the nucleotide sugar used for transfer of sialic acid onto growing oligosaccharides. Appropriate doses of sialic acid or N-acetylmannosamine for treatment of GNE myopathy are 3 to 10 grams per day, depending on the severity of the molecular lesion and the size of the patient.

Phosphoglucomutase-1 (PGM-1) deficiency is a CDG involving insufficient production of galactose, with multisystem symptoms that may include growth delay, malformations like cleft palate, hypoglycemia, and liver and heart dysfunction. Supplementary galactose at doses of 0.5 to 1 g/kg per day is used for treatment, with partial resolution of some symptoms. Uridine triacetate administered concurrently with galactose augments the therapeutic efficacy of galactose in this disease.

Several known CDG feature defects in N-glycan synthesis and processing. Type II CDG are often associated with developmental delays, hypotonia, seizures, and organ dysfunction. For these Type II CDG including CPT deficiency, N-acetylglucosamine is a potential treatment, given in doses up to 200 mg/kg/day. In these patients, uridine triacetate is co-administered with N-acetylglucosamine to enhance its efficacy in restoring N-glycan synthesis and processing.

In other glycosylation disorders in which the production, utilization, transport or availability of a pyrimidine nucleotide sugar contributes to disease pathogenesis, uridine triacetate is administered to enhance the efficacy of the particular monosaccharide deemed appropriate for that specific CDG, and the monosaccharide is administered in daily doses that are considered appropriate for it as monotherapy.

The subject that can be treated in accordance with this invention is any animal, whether vertebrate or invertebrate, but is preferably a mammalian subject including a human subject.

The invention will be better understood by reference to the following examples, which illustrate but do not limit the invention described herein.

### EXAMPLES

### Example 1

### Treatment of a CDG with Uridine Triacetate

A patient displaying developmental delays is diagnosed with a congenital disorder of glycosylation by detection of a mutation affecting glycosylation by reducing availability of a uridine-diphospho sugar, corroborated by biochemical measurements in cells from the patient. The patient is treated with oral uridine triacetate at a dose of 2 grams per square meter of body surface area, administered three times per day. The patient responds to treatment, displaying biochemical measures of improved protein glycosylation and concurrent improvements in clinical condition.

### Example 2

### GNE Myopathy

A patient displaying progressive distal muscle weakness, with vacuoles and filamentous inclusions in a muscle biopsy, is diagnosed with GNE myopathy by testing for mutations in GNE, which result in impaired synthesis of endogenous sialic acid. The patient is treated with oral N-acetylmannosamine at a dose of 3 to 10 grams per day. The patient is also treated with uridine triacetate a dosage of 2 grams per square meter of body surface area administered twice per day. The uridine triacetate improves the clinical response beyond that achieved with N-acetylmannosamine alone.

### Example 3

### Phosphoglucomutase-1 Deficiency (Type II CDG)

A patient with multisystem disease consistent with phosphoglucomutase deficiency receives a definitive diagnosis via detection of impaired transferring glycosylation and a mutation in PGM-1. The patient is treated with 1 gram/kg of d-galactose per day, and with uridine triacetate a dosage of 2 grams per square meter of body surface area administered twice per day The uridine triacetate improves the clinical response beyond that achieved with d-galactose alone.

### Example 4

### UDP-GlcNAc: Dolichol Phosphate N-acetyl-glucosamine-1 Phosphate Transferase (GPT) Deficiency (Type II CDG)

A patient displaying severe hypotonia, medically intractable seizures, developmental delay andmicrocephaly is diagnosed with a deficiency of activity of the enzyme GPT, a Type II CDG, by detection of a mutation in its encoding gene DPAGT-1. (“GPT” is the enzyme dolichol phosphate N-acetyl-glucosamine-1 phosphate transferase; “DPAGT-1” is the gene that encodes GPT.) The patient is treated with N-acetylglucosamine at a dose of 200 mg/kg per day. The patient is also treated with uridine triacetate a dosage of 2 grams per square meter of body surface area administered twice per day. The uridine triacetate improves the clinical response beyond that achieved with N-acetylglucosamine alone.

1. A method of treating a genetic glycosylation disorder in a subject, comprising administering to the subject a uridine prodrug in an amount sufficient to raise plasma uridine in the subject to a level greater than 30 micromolar.

2. The method of claim 1, wherein the genetic glycosylation disorder is a Type II congenital disorder of glycosylation.

3. The method of claim 1, wherein the genetic glycosylation disorder is a Type I congenital disorder of glycosylation or a Type III congenital disorder of glycosylation.

4. The method of claim 1, wherein the uridine prodrug is administered in one or more doses of from 0.5 to 5 grams per square meter of body surface area.

5. The method of claim 4, wherein the uridine prodrug dose is from 1.5 to 3 grams per square meter of body surface area.

6. The method of claim 1, wherein the uridine prodrug is administered in one or more doses of from 2 to 10 grams.

7. The method of claim 1, wherein the uridine prodrug is uridine triacetate.

8. The method of claim 7, wherein the uridine triacetate is administered in one, two, three, or four daily doses.

9. The method of claim 1, wherein the uridine prodrug is administered in one, two, three, or four daily doses.

10. The method of claim 1, wherein the level of plasma uridine in the subject is raised to a level of from 45 micromolar to 55 micromolar.

11. The method of claim 1, wherein the subject is a human subject.

12. The method of claim 1, further comprising administering to the subject an effective amount of a specific sugar, transfer of which is defective in the glycosylation disorder.

13. The method of claim 12, wherein the glycosylation disorder is a GNE myopathy and the specific sugar is sialic acid, mannosamine, or N-acetylmannosamine; or the glycosylation disorder is a PGM-1 deficiency and the specific sugar is d-galactose; or the glycosylation disorder is GPT deficiency and the specific sugar is N-acetylglucosamine.

14. (canceled)

15. (canceled)

16. A pharmaceutical composition for use in treating a genetic glycosylation disorder in a subject, the composition comprising a uridine prodrug in an amount sufficient to raise plasma uridine in the subject to a level greater than 30 micromolar.

17. (canceled)

18. (canceled)

19. The composition of claim 16, wherein the uridine prodrug or composition is formulated for administration in one or more doses from 0.5 to 5 grams of the uridine prodrug per square meter of body surface area.

20. The composition of claim 19, wherein the uridine prodrug dose is from 1.5 to 3 grams per square meter of body surface area.

21. (canceled)

22. The composition of claim 16, wherein the uridine prodrug is uridine triacetate.

23-28. (canceled)

29. The method of claim 4, wherein the uridine prodrug is uridine triacetate.

30. The method of claim 5, wherein the uridine prodrug is uridine triacetate.

31. The method of claim 6, wherein the uridine prodrug is uridine triacetate.
